(19) United States
(12) Patent Application Publication
(10) Pub. No.: US 2021/0063724 A1
(43) Pub. Date: Mar. 4, 2021

(54) IMAGE PICKUP APPARATUS FOR ENDOSCOPE, ENDOSCOPE, AND METHOD FOR MANUFACTURING IMAGE PICKUP APPARATUS FOR ENDOSCOPE

(71) Applicant: OLYMPUS CORPORATION, Tokyo (JP)

(72) Inventor: Kazuya MAEDA, Kamiina-gun (JP)

(73) Assignee: OLYMPUS CORPORATION, Tokyo (JP)

(21) Appl. No.: 17/008,930

(22) Filed: Sep. 1, 2020

Related U.S. Application Data
(63) Continuation of application No. PCT/JP2018/008534, filed on Mar. 6, 2018.

(51) Int. Cl. G02B 23/24; H01L 27/146; A61B 1/05; A61B 1/00

(52) U.S. Cl. CPC G02B 23/2484; G02B 23/243; H01L 27/14618; H01L 27/14623; H01L 27/14627; A61B 1/0011; H01L 27/14685; H01L 27/1469; H01L 27/14698; A61B 1/051; H01L 27/14634

(57) ABSTRACT

An image pickup apparatus for endoscope includes: an image pickup unit having a light receiving surface and a back surface; and a lens unit having a front surface and a rear surface. The light receiving surface of the image pickup unit is disposed adjacently to the rear surface of the lens unit. An entire outer surface of the lens unit except the front surface and a region of the rear surface to which the light receiving surface is disposed adjacently is covered by a light blocking layer. An entire surface of the light blocking layer is covered by a protective layer having lower moisture vapor transmission than the light blocking layer.

### CROSS REFERENCE TO RELATED APPLICATION

This application is a continuation application of PCT/JP2018/008534 filed on Mar. 6, 2018, the entire contents of which are incorporated herein by this reference.

### BACKGROUND OF THE INVENTION

### 1. Field of the Invention

The present invention relates to an image pickup apparatus for endoscope which includes an image pickup unit and a lens unit, an endoscope provided with an image pickup apparatus for endoscope which includes an image pickup unit and a lens unit, and a method for manufacturing an image pickup apparatus for endoscope which includes an image pickup unit and a lens unit.

### 2. Description of the Related Art

In an image pickup apparatus disposed on a distal end portion of an endoscope, downsizing, particularly, reduction of a diameter is important for reducing invasion.

Japanese Patent Application Laid-Open Publication No. 2012-18993 discloses, in a method for efficiently manufacturing an extremely narrow image pickup apparatus, an image pickup apparatus formed of a wafer level laminated body. A lens unit of the image pickup apparatus is prepared by a wafer level method where a bonded wafer which is formed by adhering a plurality of lens wafers, each including a plurality of lenses, to each other is cut for forming individual chips.

International Publication No. 2017/203593 discloses a method for manufacturing a lens unit where grooves are formed in a bonded wafer, a reinforcing material is filled in the grooves, a groove having a width narrower than a width of the groove is formed in the groove, and the bonded wafer is cut. With the use of the reinforcing material having a light blocking property, it is possible to efficiently manufacture a lens unit having high performance which is minimally affected by an external light.

As a method for applying disinfection and sterilization to an endoscope, autoclave treatment (high-temperature high-pressure vapor treatment) has been adopted as a mainstream. The autoclave treatment does not require a cumbersome operation, and can be readily used after sterilization. Further, a running cost of the autoclave treatment is inexpensive.

### SUMMARY OF THE INVENTION

According to an embodiment, an image pickup apparatus for endoscope includes: a lens unit having a front surface and a rear surface on a side opposite to the front surface; an image pickup unit having a light receiving surface and a back surface on a side opposite to the light receiving surface, and being provided in a state where the light receiving surface is disposed adjacently to the rear surface of the lens unit; a light blocking layer covering an entire outer surface of the lens unit except the front surface and a region of the rear surface to which the light receiving surface is disposed adjacently; and a protective layer covering an entire surface of the light blocking layer and having lower moisture vapor transmission than the light blocking layer.

According to the embodiment, an endoscope includes an image pickup apparatus for endoscope, the image pickup apparatus for endoscope including: a lens unit having a front surface and a rear surface on a side opposite to the front surface; an image pickup unit having a light receiving surface and a back surface on a side opposite to the light receiving surface, and being provided in a state where the light receiving surface is disposed adjacently to the rear surface of the lens unit; a light blocking layer covering an entire outer surface of the lens unit except the front surface and a region of the rear surface to which the light receiving surface is disposed adjacently; and a protective layer covering an entire surface of the light blocking layer and having lower moisture vapor transmission than the light blocking layer.

According to the embodiment, a method for manufacturing an image pickup apparatus for endoscope, the image pickup apparatus for endoscope including: a lens unit having a front surface and a rear surface on a side opposite to the front surface; and an image pickup unit having a light receiving surface and a back surface on a side opposite to the light receiving surface, the light receiving surface of the image pickup unit adhering to the rear surface of the lens unit, is a method including: preparing a first bonded wafer having a first main surface and a second main surface on a side opposite to the first main surface by stacking a plurality of optical element wafers each including a plurality of optical elements; preparing a second bonded wafer by adhering the light receiving surfaces of a plurality of image pickup units to the second main surface of the first bonded wafer, each of the plurality of image pickup units being formed by stacking a plurality of semiconductor elements; fixing the first main surface of the second bonded wafer to a first holding plate; dividing the first bonded wafer into a plurality of lens units by forming first grooves each having a first width along cut lines provided for forming individual image pickup apparatuses for endoscope on the first bonded wafer of the second bonded wafer; disposing a light blocking layer which covers entire outer surfaces of the plurality of lens units except the front surfaces and regions of the rear surfaces to which the light receiving surfaces respectively adhere; peeling off the first holding plate from the second bonded wafer after the back surfaces of the plurality of image pickup units of the second bonded wafer are fixed to a second holding plate; forming cutouts each having an opening of a second width wider than the first width on the front surface of the second bonded wafer along the cut lines; cutting the light blocking layer of the second bonded wafer by forming third grooves each having a third width narrower than the first width along the cut lines; disposing, on the second bonded wafer, a protective layer which covers a surface of the light blocking layer and has lower moisture vapor transmission than the light blocking layer; and forming the individual image pickup apparatuses for endoscope from the second bonded wafer by forming fourth grooves each having a fourth width narrower than the third width along the cut lines.

### BRIEF DESCRIPTION OF THE DRAWINGS

FIG. 1 is a perspective view of an endoscope system provided with an endoscope including an image pickup apparatus for endoscope according to a first embodiment;

FIG. 2 is a perspective view of the image pickup apparatus for endoscope according to the first embodiment;

FIG. 3 is a cross-sectional view of the image pickup apparatus for endoscope according to the first embodiment taken along line in FIG. 2;

FIG. 4 is a flowchart for describing a method for manufacturing the image pickup apparatus for endoscope according to the first embodiment;

FIG. 5 is a cross-sectional view for describing the method for manufacturing the image pickup apparatus for endoscope according to the first embodiment;

FIG. 6 is a cross-sectional view for describing the method for manufacturing the image pickup apparatus for endoscope according to the first embodiment;

FIG. 7 is a cross-sectional view for describing the method for manufacturing the image pickup apparatus for endoscope according to the first embodiment;

FIG. 8 is a cross-sectional view for describing the method for manufacturing the image pickup apparatus for endoscope according to the first embodiment;

FIG. 9 is a cross-sectional view for describing the method for manufacturing the image pickup apparatus for endoscope according to the first embodiment;

FIG. 10 is a cross-sectional view for describing the method for manufacturing the image pickup apparatus for endoscope according to the first embodiment;

FIG. 11 is a cross-sectional view for describing the method for manufacturing the image pickup apparatus for endoscope according to the first embodiment;

FIG. 12 is a cross-sectional view for describing the method for manufacturing the image pickup apparatus for endoscope according to the first embodiment;

FIG. 13 is a cross-sectional view for describing the method for manufacturing the image pickup apparatus for endoscope according to the first embodiment;

FIG. 14 is a cross-sectional view for describing the method for manufacturing the image pickup apparatus for endoscope according to the first embodiment;

FIG. 15 is a cross-sectional view for describing the method for manufacturing the image pickup apparatus for endoscope according to the first embodiment;

FIG. 16 is a cross-sectional view of an image pickup apparatus for endoscope according to a modification 1 of the first embodiment; and

FIG. 17 is a cross-sectional view of an image pickup apparatus for endoscope according to a second embodiment.

### DETAILED DESCRIPTION OF THE PREFERRED EMBODIMENTS

### First Embodiment

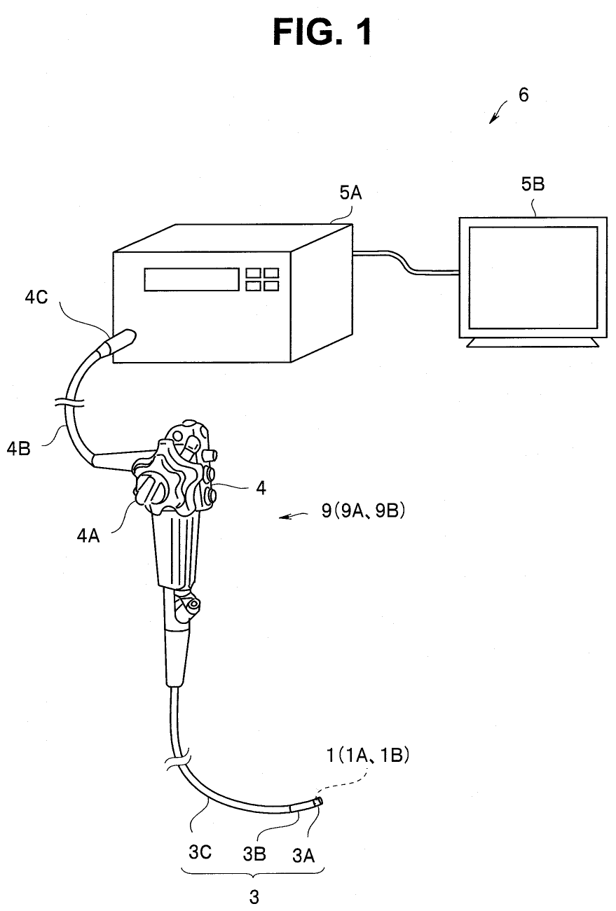

As shown in FIG. 1, an image pickup apparatus 1 for endoscope according to an embodiment (hereinafter, also referred to as “image pickup apparatus 1”) is disposed in an endoscope 9 of an endoscope system 6.

In a description made hereinafter, drawings based on respective embodiments are schematic views. Accordingly, it must be noted that a relationship between a thickness and a width of each portion, a ratio between thicknesses and relative angles of respective portions differ from the corresponding relationships of portions of an actual image pickup apparatus. There may be a case where portions of the image pickup apparatus are described with different size relationship or different ratios between the drawings. The illustration of some components may be omitted. An object direction (a Z-axis value increasing direction in the drawings) is set as an upward direction.

The endoscope 9 includes: an insertion section 3; a grasping portion 4 disposed on a proximal end portion side of the insertion section 3; a universal cord 4B extending from the grasping portion 4; and a connector 4C disposed on a proximal end portion of the universal cord 4B. The insertion section 3 includes: a distal end portion 3A; a bending portion 3B extending from the distal end portion 3A and being bendable so as to change a direction of the distal end portion 3A; and a flexible portion 3C extending from the bending portion 3B. The image pickup apparatus 1 is disposed on the distal end portion 3A. A rotatable angle knob 4A which is an operation section for allowing a surgeon to operate the bending portion 3B is disposed on the grasping portion 4.

The universal cord 4B is connected to a processor 5A via the connector 4C. The processor 5A controls an entirety of the endoscope system 6, and applies signal processing to an image pickup signal which the image pickup apparatus 1 outputs and outputs the image pickup signal as an image signal. A monitor 5B displays the image signal which the processor 5A outputs as an endoscope image. Although the endoscope 9 is a flexible endoscope, the endoscope 9 may be a rigid endoscope. In other words, the flexible portion and the like are not indispensable components of the endoscope according to the embodiment. The endoscope 9 may be a capsule endoscope, and may be used in a medical field or in an industrial field.

As described later, a side surface of the image pickup apparatus 1 is covered not only by a light blocking layer 30 but also by a protective layer 40 having low moisture vapor transmission (see FIG. 3). Accordingly, the endoscope 9 has high performance and high reliability.

### <Configuration of Image Pickup Apparatus>

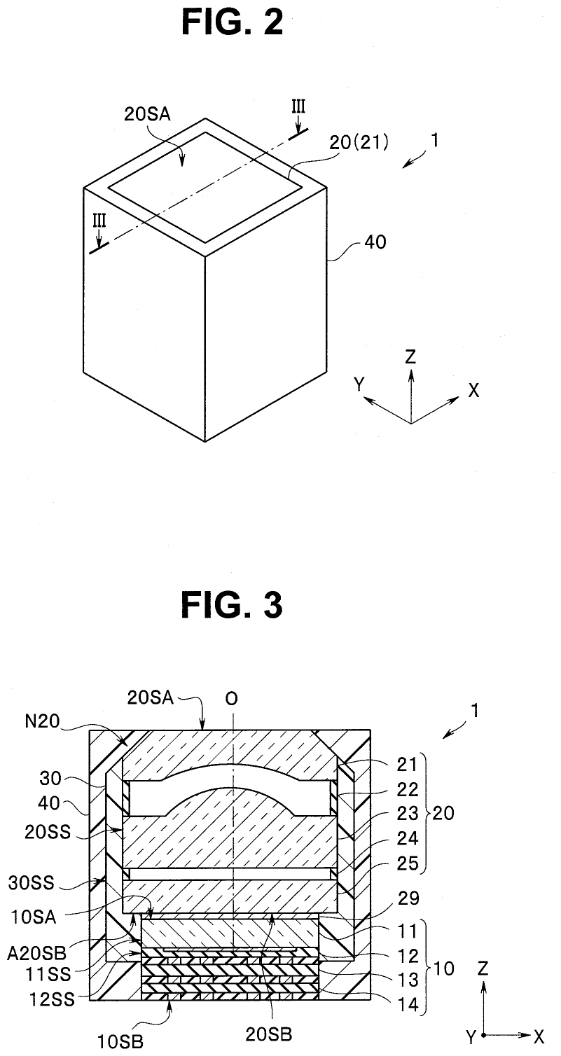

As shown in FIG. 2 and FIG. 3, the image pickup apparatus 1 includes an image pickup unit 10 and a lens unit 20. The image pickup unit 10 has a light receiving surface 10SA and a back surface 10SB on a side opposite to the light receiving surface 10SA. The lens unit 20 has a front surface 20SA on which light is incident and a rear surface 20SB on a side opposite to the front surface 20SA.

The image pickup unit 10 is a wafer level stacked body where a cover glass 11 and a plurality of semiconductor elements (an imager 12, semiconductor elements 13, 14) are stacked. Although not shown, the plurality of stacked semiconductor elements are connected with each other through respective through wirings, and connection electrodes are disposed on the back surface 10SB.

The imager 12 is a CCD element or a CMOS element. The imager 12 may be a surface irradiation type image sensor or a back surface irradiation type image sensor.

The semiconductor elements 13, 14 apply primary processing to an image pickup signal which the imager 12 outputs or process a control signal which controls the imager 12. For example, the semiconductor elements 13, 14 each include an AD converter, a memory, a transmission output circuit, a filter circuit, a thin film capacitor, and a thin film inductor. The image pickup unit 10 includes at least the cover glass 11 and the imager 12.

The lens unit 20 is a wafer level stacked body where a plurality of optical elements 21 to 25 are stacked. The lens unit 20 forms an image on the light receiving portion 12A of the imager 12 by condensing light incident from the front surface 20SA. The optical element 21 is positioned on a frontmost portion of the lens unit 20, and is a planoconcave lens having the front surface 20SA. A cutout N20 having a picture frame shape is formed on an outer periphery of the front surface 20SA of the optical element 21. The optical elements 22, 25 are each a spacer having a through hole which forms an optical path at a center of the spacer. The optical element 23 is a planoconvex lens. The optical element 25 is an infrared cut filter element. The optical elements 21, 23 are each, for example, a resin molded element or a hybrid lens element where a resin lens is disposed in a parallel flat plate glass.

Although not shown, the lens unit 20 also includes an adhesive layer and other optical components such as a flare aperture and a brightness aperture. A configuration of the lens unit 20 is not limited to the configuration used in the image pickup apparatus 1, and the configuration such as the number of resin lenses, spacers, and apertures is suitably selected corresponding to a specification of an image pickup apparatus.

In the image pickup apparatus 1, the light receiving surface 10SA of the image pickup unit 10 adheres to the rear surface 20SB of the lens unit 20 by a transparent adhesive layer 29. A size of the rear surface 20SB is larger than a size of the light receiving surface 10SA and hence, an outer peripheral region A20SB having a picture frame shape to which the light receiving surface 10SA does not adhere is formed on the rear surface 20SB of the lens unit 20.

A relative size relationship between the lens unit 20 and the image pickup unit 10 is not limited to the above-mentioned relationship, and the light receiving surface 10SA and the rear surface 20SB may have the same size, or the size of the light receiving surface 10SA may be larger than the size of the rear surface 20SB.

Besides side surfaces 20SS of the lens unit 20 and side surfaces of the image pickup unit 10, the outer peripheral region A20SB and the transparent adhesive layer 29 are also covered by the light blocking layer 30. An entire surface 30SS of the light blocking layer 30 is covered by the protective layer 40 having lower moisture vapor transmission than the light blocking layer 30. In other words, in the image pickup apparatus 1, the outer peripheral region A20SB, all side surfaces, and the cutout N20 having a picture frame shape formed on the front surface 20SA are covered by the protective layer 40.

In the image pickup apparatus 1 easily manufactured by a wafer level method, the outer peripheral region A20SB and the transparent adhesive layer 29 are also covered by the light blocking layer 30 and hence, the image pickup apparatus 1 is minimally affected by an external light and has high performance. Moisture vapor transmission of a reinforcing material having light blocking property is not low. Accordingly, in autoclave treatment, moisture which permeates the reinforcing material enters the inside of a lens unit, and forms a fog on a lens or lowers an adhesive strength between the lenses thus giving rise to a possibility that reliability of an image pickup apparatus is lowered. In the image pickup apparatus 1, the entire surface of the light blocking layer 30 is covered by the protective layer 40 and hence, the intrusion of water into the image pickup apparatus 1 is prevented whereby the image pickup apparatus 1 has high reliability. Accordingly, the endoscope 9 having the image pickup apparatus 1 has high performance and high reliability.

The cutout N20 is formed on the outer periphery of the front surface 20SA of the optical element 21 and hence, a boundary portion between the light blocking layer 30 and the optical element 21 is also covered by the protective layer 40. Accordingly, intrusion of water into the image pickup apparatus from the boundary portion is also prevented.

In the image pickup apparatus 1, the image pickup unit 10 is a stacked chip formed by stacking the cover glass 11 and the plurality of semiconductor elements 12 to 14 including the imager 12, and side surfaces of the cover glass 11 and side surfaces of the imager 12 are covered by the light blocking layer 30. Accordingly, the image pickup apparatus 1 is not affected by an external light which is incident from the side surfaces of the cover glass 11 and the side surfaces of the imager 12 and hence, the image pickup apparatus 1 has particularly high performance.

### <Method for Manufacturing Image Pickup Apparatus>

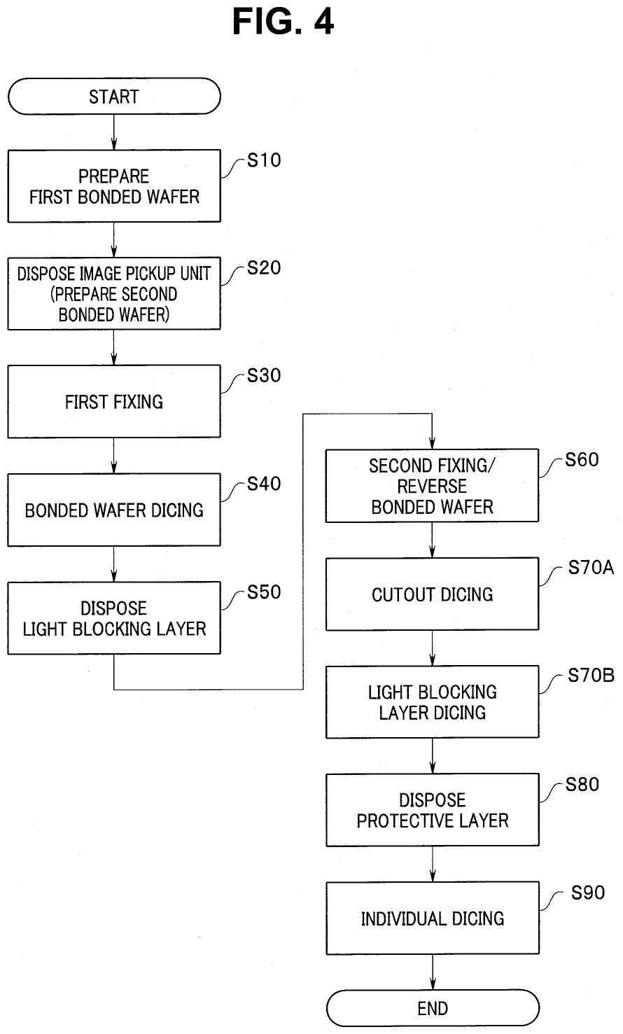

Next, a method for manufacturing the image pickup apparatus according to the embodiment is described in accordance with a flowchart shown in FIG. 4.

### <Step S10> First Bonded Wafer Preparing Step

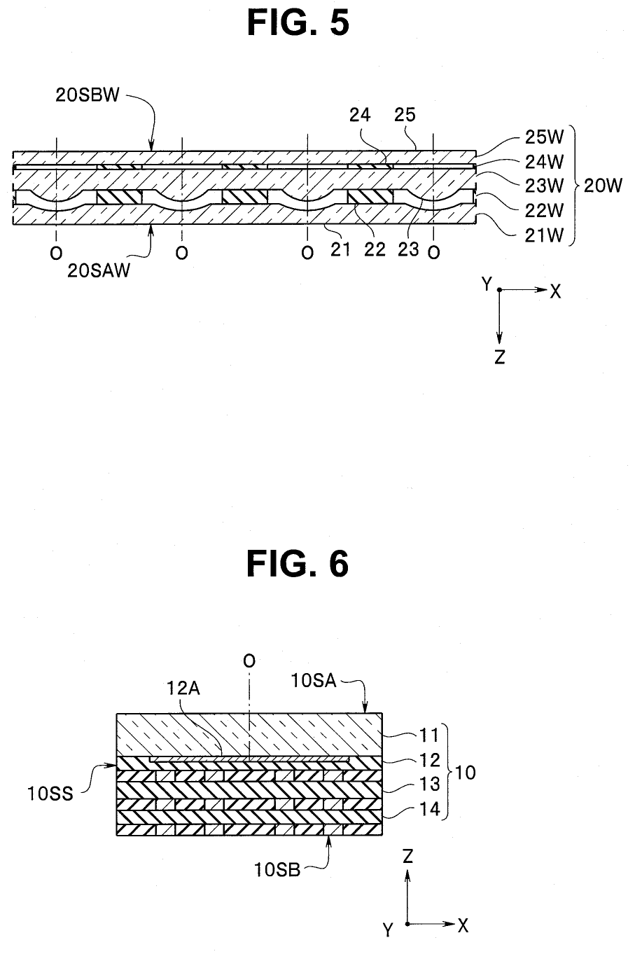

As shown in FIG. 5, the lens unit 20 is a wafer level stacked body manufactured such that a first bonded wafer 20W is prepared by stacking a plurality of optical element wafers 21W to 25W and making such optical element wafers 21W to 25W adhere to each other, and cutting the first bonded wafer 20W into individual chips. Accordingly, four side surfaces 20SS of the lens unit 20 are cut surfaces.

The lens unit 20 is a rectangular parallelepiped body. However, the lens unit 20 may be a polygonal prism where corner portions of side surfaces are chamfered in a straight shape or an approximately rectangular parallelepiped body where corner portions are chamfered in a curved shape.

First, the plurality of optical element wafers 21W to 25W respectively including a plurality of optical elements 21 to 25 are prepared. For example, the optical element wafers 21W, 23W where a plurality of lenses are arranged in a matrix array are prepared by arranging resin lenses on a parallel flat plate glass wafer which is a base body. The resin lenses may be preferably formed using an energy curing type resin. It is sufficient that the parallel flat plate glass wafer be transparent in a wavelength band of light used for image pickup. For example, the parallel flat plate glass wafer is formed using borosilicate glass, quartz glass or single crystal sapphire.

When an energy curing type resin receives an energy such as heat, an ultraviolet ray, an electron beam or the like from the outside, a crosslinking reaction or a polymerization reaction advances. A curing type resin is, for example, a transparent resin such as an ultraviolet curing type silicone resin, an epoxy resin or an acrylic resin. In the embodiment, “transparent” means that absorption and scattering of light in a material are small to an extent that the resin can withstand the use within a use wavelength range.

An ultraviolet curing type resin which is uncured and in a liquid form or in a gel form is disposed on the glass wafer, and in a state where a mold having a predetermined inner surface shape is pressed to the glass wafer, an ultraviolet ray is irradiated to the ultraviolet curing type resin so that the resin is cured whereby the resin lenses are prepared. To enhance an interface adhesive strength between the glass and the resin, it is preferable to apply silane coupling treatment or the like to the glass wafer before the resin is disposed on the glass wafer. An outer surface shape of the resin lens is formed by transferring the inner surface shape of the mold and hence, an aspheric lens can be easily prepared.

The optical element wafers 22W, 24W which constitute spacers are prepared by forming a plurality of through holes in a silicon wafer, for example, using an etching method.

The element wafer 25W is a filter wafer made of an infrared cutting material which removes undesired infrared rays (for example, light having a wavelength of 700 nm or more). As the filter wafer, it may be possible to use a flat plate glass wafer, on a surface of which a band pass filter which allows transmission of only light having a predetermined wavelength and cuts lights having undesired wavelengths or the like is disposed.

As shown in FIG. 5, the plurality of optical element wafers 21W to 25W respectively including the plurality of optical elements 21 to 25 are stacked in a state where respective optical axes O of the optical elements 21 to 25 are aligned with each other. The first bonded wafer 20W having a first main surface 20SAW and a second main surface 20SBW on a side opposite to the first main surface 20SAW is prepared. As described later, the first main surface 20SAW forms the front surface 20SA of the image pickup apparatus 1, and the second main surface 20SBW forms a rear surface 20SB of the image pickup apparatus 1.

### <Step S20> Image Pickup Unit Disposing Step (Second Bonded Wafer Preparing Step)

Although not shown, the image pickup unit 10 shown in FIG. 6 is also the same wafer level image pickup body as the lens unit 20. Accordingly, the cover glass 11 and the semiconductor elements 12 to 14 have the same size (an outer size in a direction orthogonal to an optical axis), and four side surfaces 10SS are cut surfaces. FIG. 5 and FIG. 6 are reversed in a vertical direction (Z axis direction).

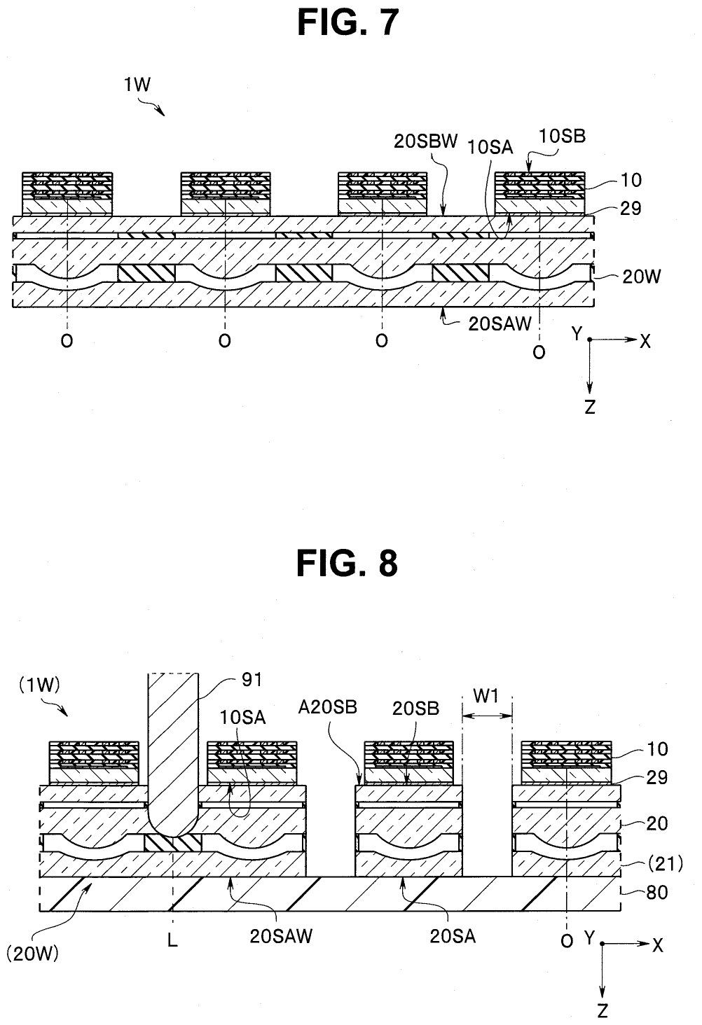

As shown in FIG. 7, a second bonded wafer 1W is prepared by making the light receiving surfaces 10SA of a plurality of image pickup units 10 adhere to the second main surface 20SBW of the first bonded wafer 20W by transparent adhesive layers 29. The adhesive layer 29 is made of an ultraviolet curing type resin, for example, an epoxy resin, a polyimide, BCB (benzocyclobutene) resin, or a silicone-based resin.

In other words, the second bonded wafer 1W includes the first bonded wafer 20W, the plurality of image pickup units 10, and the adhesive layers 29. The optical axes O of the image pickup units 10 are aligned with the optical axes O of the first bonded wafer 20W.

In the manufacturing method of the embodiment, the image pickup units 10 are prepared as separate members from the first bonded wafer 20W. Accordingly, an operation checking step of the image pickup units 10 is performed before the first bonded wafer preparing step and hence, only the image pickup units 10 which are found as a non-defective product by checking are made to adhere to the first bonded wafer 20W. Accordingly, the manufacturing method of the embodiment exhibits a favorable yield.

### <Step S30> First Fixing Step

The first fixing step for fixing the first main surface 20SAW of the second bonded wafer 1W to a first holding plate 80 is performed. The first holding plate 80 is, for example, a dicing tape for temporarily fixing a work in a dicing step. Step S30 may be performed prior to step S20.

### <Step S40> Bonded Wafer Dicing Step

As shown in FIG. 8, first grooves each having a first width W1 which penetrates the second bonded wafer 1W are formed along cut lines L provided for forming individual image pickup apparatuses 1 for endoscope using a first dicing saw 91. As a result, the first bonded wafer 20W is cut into a plurality of lens units 20.

In performing such cutting, provided that the penetration groove having a predetermined width can be formed, laser dicing or plasma dicing may be used. Alternatively, an etching method or the like may be used in place of mechanical working.

The first width W1 is narrower than a distance between the image pickup units 10 disposed adjacently to each other. In other words, a size (the outer size in a direction orthogonal to the optical axis) of the rear surface 20SB of the lens unit 20 is larger than a size (the outer size in the direction orthogonal to the optical axis) of the light receiving surface 10SA of the image pickup unit 10. Accordingly, the outer peripheral region A20SB having a picture frame shape to which the light receiving surface 10SA of the image pickup unit 10 does not adhere is formed on the rear surface 20SB of the lens unit 20.

### <Step S50> Light Blocking Layer Disposing Step

In the second bonded wafer 1W, the light blocking layer 30 is disposed such that the light blocking layer 30 covers the outer peripheral regions A20SB of the plurality of lens units 20 and the side surfaces 20SS of the plurality of lens units 20.

In the manufacturing method of the embodiment, portions of a light blocking resin 30R each disposed in the first groove having the first width W1 are the light blocking layers 30.

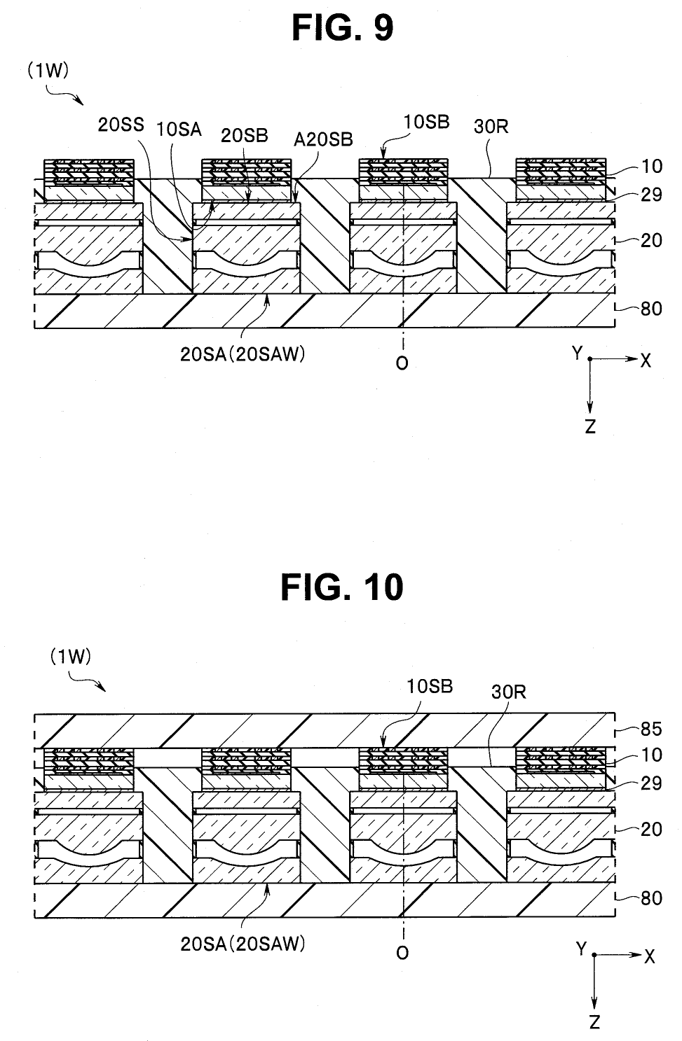

In other words, first, as shown in FIG. 9, the light blocking resin 30R is disposed in the groove having the first width W1, and thermal curing treatment is applied to the light blocking resin 30R. The light blocking resin 30R is disposed such that the groove having the first width W1 is not completely filled with the light blocking resin 30R. However, the light blocking resin 30R is disposed so as to cover at least the side surfaces 11S of the cover glass 11 and the side surfaces 12SS of the imager 12 (see FIG. 3).

Note that after the groove having the first width W1 is completely filled with the light blocking resin 30R, for example, etching treatment may be applied to the light blocking resin 30R such that side surfaces of the semiconductor element 14 which constitutes the back surface 10SB are exposed. Alternatively, after the groove having the first width W1 is completely filled with the light blocking resin 30R, an upper portion of the light blocking resin 30R in the groove having the first width W1 may be removed using a dicing saw. In this case, a cutout may be formed on the side surfaces of the semiconductor element 14.

By performing step 50 (light blocking layer disposing step) after step S60 (second fixing step/bonded wafer reversing step) described later, the light blocking resin 30R can be disposed in a state where the groove is not completely filled with the light blocking resin 30R. In other words, in the second fixing step for fixing the back surfaces 10SB of the plurality of image pickup units 10 of the second bonded wafer 1W to a second holding plate 85, a dicing tape having a thick adhesive layer is used as the second holding plate 85. As a result, portions of the side surfaces of the image pickup unit 10 are also covered by the adhesive layer of the dicing tape. Accordingly, the groove having the first width W1 is not completely filled with the light blocking resin 30R.

In a description made hereinafter, a step where the side surfaces of the semiconductor element 14 are exposed by applying etching treatment to the light blocking resin 30R is described.

The light blocking resin 30R is, for example, a thermosetting resin which contains particles having light blocking property. For example, cut margins, that is, the inside of the groove having the first width W1 and a space formed between the image pickup units 10 disposed adjacently to each other are filled with an epoxy resin in which carbon particles are dispersed by an inkjet method. The particles having light blocking property may be black titanium oxide particles or the like. The resin may be a polyimide, BCB (benzocyclobutene) resin, or a silicone-based resin.

The particles having light blocking property such as carbon particles have hydrophilicity and hence, a resin which contains particles having light blocking property has high moisture vapor transmission compared to a resin which does not contain particles having light blocking property. In other words, when hydrophilic particles having hydrophilicity are dispersed in a resin, moisture vapor transmission is increased.

### <Step S60> Second Fixing Step/Bonded Wafer Reversing Step

As shown in FIG. 10, a second fixing step for fixing the back surfaces 10SB of the plurality of image pickup units 10 of the second bonded wafer 1W to the second holding plate 85 is performed. The second holding plate 85 is, for example, a dicing tape.

The groove having the first width W1 is not completely filled with the light blocking resin 30R and hence, a gap exists between the light blocking resin 30R and the second holding plate 85.

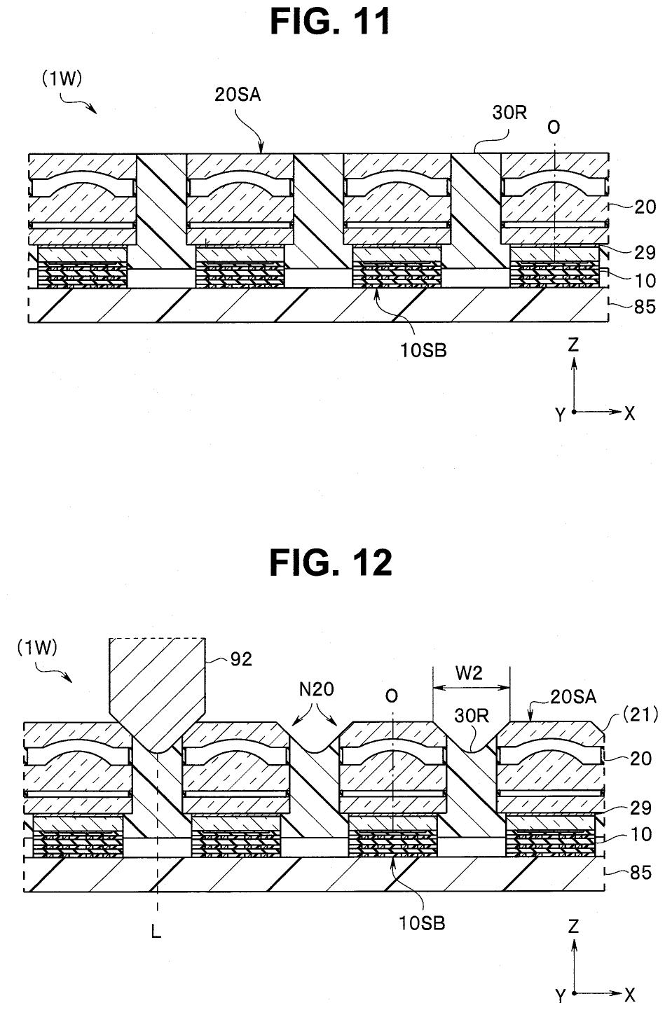

As shown in FIG. 11, the first holding plate 80 is peeled off from the front surfaces 20SA of the plurality of lens units 20 which are the first main surface 20SAW of the second bonded wafer 1W. At this stage of operation, the back surfaces 10SB of the image pickup units 10 are fixed to the second holding plate 85. Accordingly, the plurality of lens units 20 are fixed at predetermined positions.

FIG. 11 is reversed in the vertical direction (Z axis increasing direction) with respect to FIG. 10. In other words, the second bonded wafer 1W is held in such a manner that the back surfaces 10SB of the image pickup units 10 on a side opposite to the front surfaces 20S of the lens units 20 are fixed to the second holding plate 85.

A tack strength of a dicing tape is decreased due to ultraviolet ray irradiation treatment or heat treatment. To decrease a tack strength of the first holding plate 80 while maintaining a tack strength of the second holding plate 85, it is preferable that the first holding plate 80 and the second holding plate 85 be formed of different kinds of dicing tapes where the respective tack strengths are decreased by different treatments. For example, a first dicing tape where a tack strength is decreased by irradiation of an ultraviolet ray is used as the first holding plate 80, and a second dicing tape where a tack strength is decreased by heat treatment is used as the second holding plate 85. It goes without saying that the first holding plate 80 may be formed of the second dicing tape, and the second holding plate 85 may be formed of the first dicing tape. Further, both the first holding plate and the second holding plate may be formed of the same kind of dicing tape.

### <Step S70A> Cutout Dicing Step

As shown in FIG. 12, cutouts N20 having a picture frame shape are formed on the second bonded wafer 1W along the cut lines L provided for forming the individual image pickup apparatuses 1 for endoscope. In other words, using a second dicing saw 92, the cutout N20 with an opening having a second width W2 wider than the first width W1 is formed on the front surface 20SA.

Provided that outer peripheries of the respective front surfaces 20SA of the plurality of optical elements 21 at an uppermost portion (a position closest to an object) of the second bonded wafer 1W are cut out in a picture frame shape, a cross-sectional shape of the cutout N20 may be a rectangular shape having side surfaces orthogonal to the front surface 20SA. The cutout N20 is formed by cutting out only the optical element 21, and an adhesive layer (not shown) which makes the optical element 21 and the optical element 22 adhere to each other is not cut out.

### <Step S70B> Light Blocking Layer Dicing Step

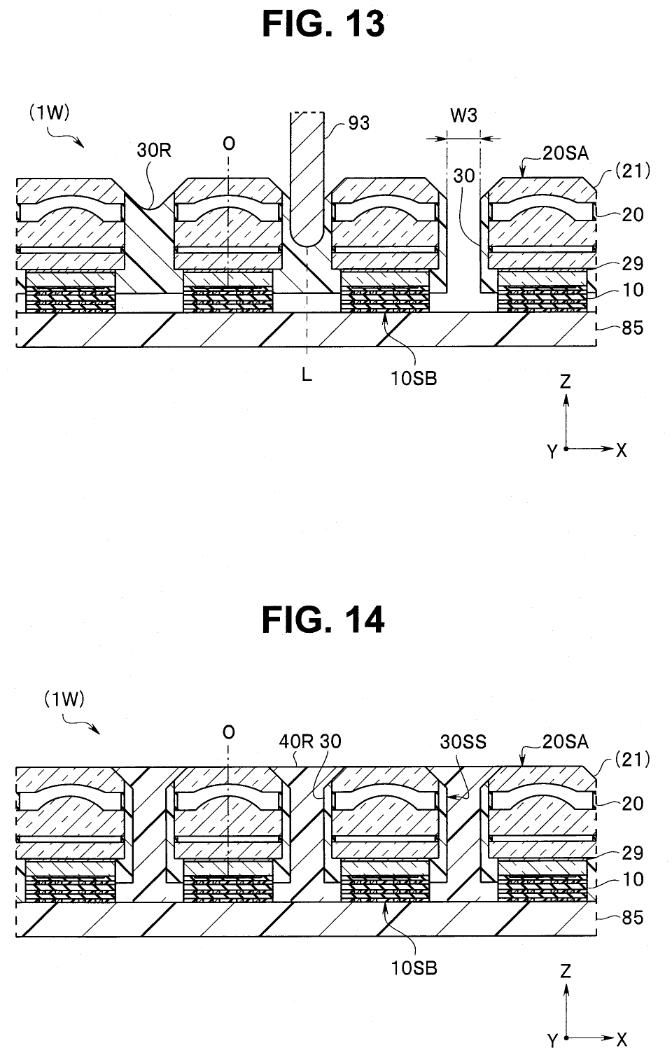

As shown in FIG. 13, third grooves each having a third width W3 which is narrower than the first width W1 are formed along the cut lines L provided for forming the individual image pickup apparatuses 1 for endoscope using a third dicing saw 93, and thereby the light blocking resin 30R is cut.

When the light blocking resin 30R is diced, the light blocking resin 30R forms the light blocking layers 30 each disposed on the side surfaces of the image pickup apparatus. A thickness of the light blocking layer 30 is (first width W1-third width W3)/2.

A thickness of the light blocking layer 30 on the side surfaces of the lens unit 20 is preferably 5 μm or more, and is more preferably 10 μm or more. Provided that the thickness of the light blocking layer 30 falls within the above-mentioned range or more, light blocking property is ensured. An upper limit of the thickness of the light blocking layer 30 is, for example, 100 μm or less.

Step S70B may also be performed before step S70A.

### <Step S80> Protective Layer Disposing Step

The protective layers 40 each covering the entire surface 30SS of the light blocking layer 30 and has lower moisture vapor transmission than the light blocking layer 30 are disposed. The protective layer 40 is a moisture proof film and prevents the permeation of water into the light blocking layer 30. In the manufacturing method of the embodiment, portions of a resin 40R each disposed in the third groove are the protective layers 40.

As shown in FIG. 14, the third groove is filled with the resin 40R which constitutes the protective layer 40. A space between the light blocking resin 30R and the second holding plate 85 is also filled with the resin 40R.

Since an adhesive strength between the protective layer 40 and the light blocking layer 30 is high, the resin (first resin) for forming the light blocking resin 30R and the resin (second resin) 40R for forming the protective layer 40 may be the same thermosetting resin.

In other words, the protective layer 40 may be made of the first resin, and the light blocking layer 30 may be made of the first resin in which particles having light blocking property are dispersed.

### <Step S90> Individual Dicing Step

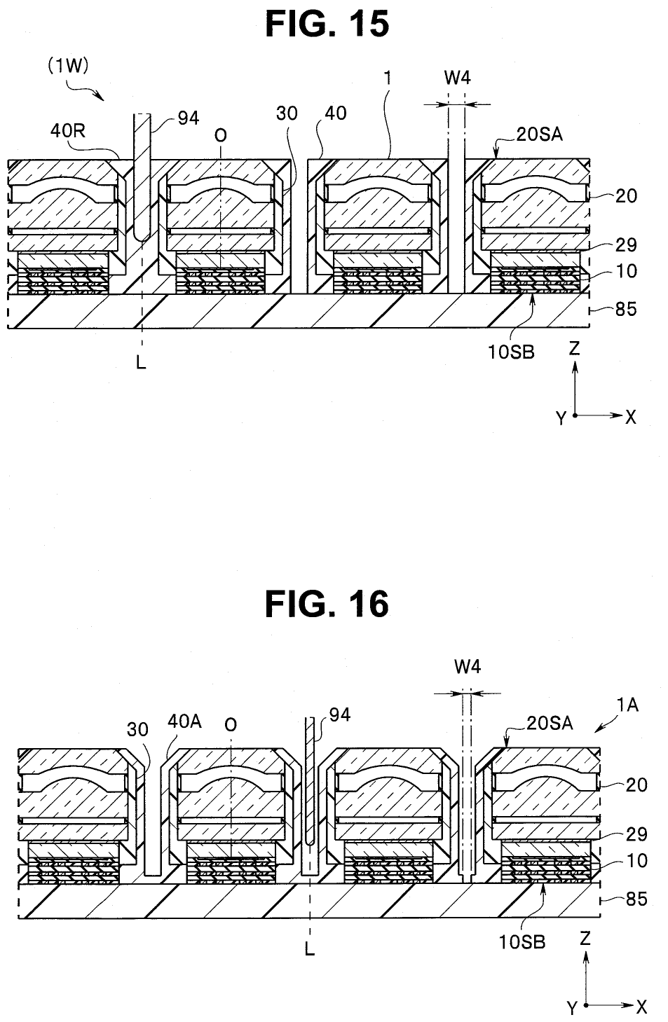

As shown in FIG. 15, by forming fourth grooves each having a fourth width W4 narrower than the third width W3 along the cut lines L using a fourth dicing saw 94, the second bonded wafer 1W is cut thus forming the individual image pickup apparatuses 1 for endoscope.

When the resin 40R is diced, the resin 40R forms the protective layers 40 each covering the entire surface of the light blocking layer 30 of the image pickup apparatus 1. A thickness of the protective layer 40 is (third width W3-fourth width W4)/2.

A thickness of the protective layer 40 is preferably 5 μm or more, and more preferably 10 μm or more. Provided that the thickness of the protective layer 40 falls within the above-mentioned range or more, moisture proof property is ensured. An upper limit of the thickness of the protective layer 40 is, for example, 100 μm or less.

It is particularly preferable that the protective layer 40 of the endoscope 9 to which autoclave treatment is applied have moisture vapor transmission of 5 g/(m²×day) or less in a moisture vapor transmission test prescribed in JIS Z 0208.

According to the manufacturing method of the embodiment, the image pickup apparatus for endoscope which is minimally affected by an external light thus having high performance, and can prevent the intrusion of water into the lens unit 20 thus having high reliability can be easily manufactured by a wafer level method.

### Second Embodiment

An image pickup apparatus 1A, an endoscope 9A, and a method for manufacturing the image pickup apparatus 1A according to a second embodiment are described. The image pickup apparatus 1A is substantially equal to the image pickup apparatus 1 and acquires the same advantageous effects and hence, components identical with the corresponding components of the image pickup apparatus 1 are given with the same symbols, and the description of such components is omitted.

A protective layer 40A of the image pickup apparatus 1A shown in FIG. 16 is formed as a protective layer which covers light blocking layers 30 before individual dicing step (S90). In the individual dicing step, the protective layer 40A on a bottom surface of a groove is cut.

The protective layer 40A is a polyparaxylene film, a silicon nitride film, or a silicon oxide film disposed by a vacuum film forming method. In other words, a side surface of the protective layer 40A may not be a cut surface of a resin 40R.

With the use of a vacuum film forming method, the protective layer 40A having lower moisture vapor transmission than the protective layer 40 formed by cutting the resin 40R can be easily disposed. When the protective layer 40A is transparent, the protective layer 40A may cover a front surface 20SA of the lens unit 20.

### Third Embodiment

An image pickup apparatus 1B, an endoscope 9B, and a method for manufacturing the image pickup apparatus 1B according to the third embodiment are described. The image pickup apparatus 1B is substantially equal to the image pickup apparatuses 1, 1A and acquires the same advantageous effects and hence, components identical with the corresponding components of the image pickup apparatuses 1, 1A are given with the same symbols, and the description of such components is omitted.

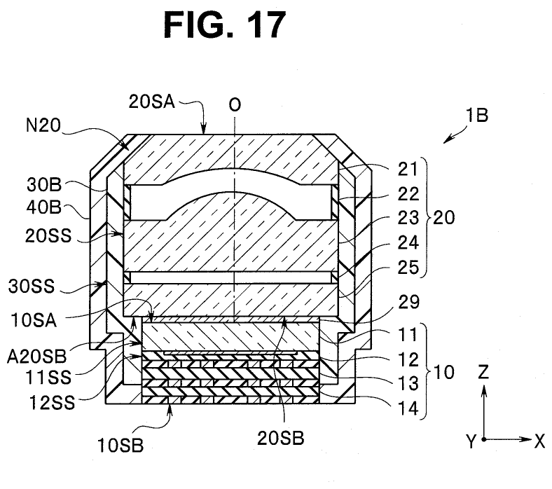

A light blocking layer 30B of the image pickup apparatus 1B shown in FIG. 17 is a light blocking film formed in a groove before light blocking layer dicing step (S70B). A protective layer 40B is also formed as a protective layer which covers the light blocking layers 30 before individual dicing step (S90).

The light blocking layer 30B is, for example, a metal film, for example, a copper film formed by electroless plating. The metal film may have pin holes. However, the light blocking layer 30B is covered by the protective layer 40B and hence, the image pickup apparatus 1B has high performance and high reliability.

It goes without saying that the endoscopes 9A, 9B provided with the image pickup apparatus 1A, 1B on a distal end portion acquires advantageous effects which the endoscope 9 provided with the image pickup apparatus 1 has, and advantageous effects of each image pickup apparatus 1A, 1B.

The present invention is not limited to the above-mentioned embodiments and the like, and various modifications and alterations are conceivable without departing from the gist of the present invention.

What is claimed is:

1. An image pickup apparatus for endoscope, comprising:
a lens unit having a front surface and a rear surface on a side opposite to the front surface;
an image pickup unit having a light receiving surface and a back surface on a side opposite to the light receiving surface, and being provided in a state where the light receiving surface is disposed adjacently to the rear surface of the lens unit;
a light blocking layer covering an entire outer surface of the lens unit except the front surface and a region of the rear surface to which the light receiving surface is disposed adjacently; and
a protective layer covering an entire surface of the light blocking layer and having lower moisture vapor transmission than the light blocking layer.

2. The image pickup apparatus for endoscope according to claim 1, wherein a side surface of the lens unit is a cut surface.

3. The image pickup apparatus for endoscope according to claim 2, wherein the image pickup unit is a stacked chip where a plurality of semiconductor elements and a cover glass are stacked, the plurality of semiconductor elements including an imager, and
a side surface of the cover glass and a side surface of the imager are covered by the light blocking layer.

4. The image pickup apparatus for endoscope according to claim 2, wherein the protective layer is made of a first resin, and
the light blocking layer is made of a light blocking resin where particles having light blocking property are dispersed in the first resin.

5. The image pickup apparatus for endoscope according to claim 2, wherein the rear surface of the lens unit is larger than the light receiving surface of the image pickup unit, and
an outer peripheral region of the rear surface to which the light receiving surface is not disposed adjacently is covered by the light blocking layer.

6. An endoscope comprising an image pickup apparatus, the image pickup apparatus including:
a lens unit having a front surface and a rear surface on a side opposite to the front surface;
an image pickup unit having a light receiving surface and a back surface on a side opposite to the light receiving surface, and being provided in a state where the light receiving surface is disposed adjacently to the rear surface of the lens unit;
a light blocking layer covering an entire outer surface of the lens unit except the front surface and a region of the rear surface to which the light receiving surface is disposed adjacently; and
a protective layer covering an entire surface of the light blocking layer and having lower moisture vapor transmission than the light blocking layer.

7. A method for manufacturing an image pickup apparatus for endoscope, the image pickup apparatus for endoscope including: a lens unit having a front surface and a rear surface on a side opposite to the front surface; and an image pickup unit having a light receiving surface and a back surface on a side opposite to the light receiving surface, the light receiving surface of the image pickup unit adhering to the rear surface of the lens unit, the method comprising:
preparing a first bonded wafer having a first main surface and a second main surface on a side opposite to the first main surface by stacking a plurality of optical element wafers each including a plurality of optical elements;
preparing a second bonded wafer by adhering the light receiving surfaces of a plurality of image pickup units to the second main surface of the first bonded wafer, each of the plurality of image pickup units being formed by stacking a plurality of semiconductor elements;
fixing the first main surface of the second bonded wafer to a first holding plate;
dividing the first bonded wafer into a plurality of lens units by forming first grooves each having a first width along cut lines provided for forming individual image pickup apparatuses for endoscope on the first bonded wafer of the second bonded wafer;
disposing a light blocking layer which covers entire outer surfaces of the plurality of lens units except the front surfaces and regions of the rear surfaces to which the light receiving surfaces respectively adhere;
peeling off the first holding plate from the second bonded wafer after the back surfaces of the plurality of image pickup units of the second bonded wafer are fixed to a second holding plate;
forming cutouts each having an opening of a second width wider than the first width on the front surface of the second bonded wafer along the cut lines;
cutting the light blocking layer of the second bonded wafer by forming third grooves each having a third width narrower than the first width along the cut lines;
disposing, on the second bonded wafer, a protective layer which covers a surface of the light blocking layer and has lower moisture vapor transmission than the light blocking layer; and
forming the individual image pickup apparatuses for endoscope from the second bonded wafer by forming fourth grooves each having a fourth width narrower than the third width along the cut lines.

8. The method for manufacturing the image pickup apparatus for endoscope according to claim 7, wherein
the image pickup unit is a stacked chip formed by stacking the plurality of semiconductor elements and a cover glass, the plurality of semiconductor elements including an imager, and
a side surface of the cover glass and a side surface of the imager are covered by the light blocking layer when the light blocking layer is disposed.

9. The method for manufacturing the image pickup apparatus for endoscope according to claim 7, wherein
the protective layer is made of a first resin, and
the light blocking layer is made of the first resin in which particles having light blocking property are dispersed.

10. The method for manufacturing the image pickup apparatus for endoscope according to claim 7, wherein
the rear surface of the lens unit is larger than the light receiving surface of the image pickup unit, and
an outer peripheral region of the rear surface to which the light receiving surface does not adhere is covered by the light blocking layer.

11. The method for manufacturing the image pickup apparatus for endoscope according to claim 7, wherein the protective layer is disposed by a vacuum film forming method.
